(19) United States
(12) Patent Application Publication
(10) Pub. No.: US 2021/0279861 A1
(43) Pub. Date: Sep. 9, 2021

(54) A METHOD OF PROCESSING IMAGES

(71) Applicant: UNIVERSITY COLLEGE DUBLIN, NATIONAL UNIVERSITY OF IRELAND, DUBLIN, Dublin (IE)

(72) Inventor: Peter Joseph MACMAHON, Dublin (IE)

(21) Appl. No.: 16/347,544

(22) PCT Filed: Nov. 2, 2017
(86) PCT No.: PCT/EP2017/078047

§ 371 Date: May 3, 2019

(30) Foreign Application Priority Data
Nov. 4, 2016 (GB) 1618645.4

(51) Int. Cl. G06T 7/00; A61B 6/00; G06T 5/50

(52) U.S. Cl. CPC G06T 7/0012; A61B 6/481; A61B 6/504; G06T 2207/30101; G06T 2207/10081; G06T 2207/20224; G06T 2207/30016; G06T 5/50

(57) ABSTRACT

A method of processing cross-sectional post contrast images. The method comprises obtaining a first cross-sectional post contrast image of at least part of a body (70). The first cross-sectional post contrast image represents a first time point. A second cross-sectional post contrast image of the at least part of the body is also obtained (72). The second cross-sectional post contrast image represents a second time point which is different from the first time point. A first difference image is generated using the first and second cross-sectional post contrast images (74). The first difference image highlights any differences between the first and second cross-sectional post contrast images.

The present invention is directed towards a method of processing images, and in particular towards a method of processing cross-sectional post contrast images. Image processing systems and computer-readable media for performing the methods are also provided.

Acute ischaemic strokes are medical conditions which occur when vessels supplying part of the brain have become occluded, and thus that part of the brain is at risk of dying. The best treatment for such conditions is the urgent re-opening of the occluded vessel or vessels. A common therapy for acute ischaemic strokes is to supply a thrombolysis medication, which is otherwise known as ‘clot-busting’ medication. This thrombolysis medication would be inappropriate and potentially damaging for a patient who is suffering from a different kind of stroke such as a haemorrhagic stroke, or a very large ischaemic stroke as it can cause bleeding in the brain in these circumstances. Therefore, it is important to correctly identify the stroke before administering medication. It is also understandably important to identify patients suffering from acute ischaemic stroke quickly so that the medication, or a more advanced therapy such as thrombectomy, can be promptly administered.

Post contrast computed tomography (CT) angiography is an existing technique used to visualise arterial and venous vessels throughout the body, and has a particular application in the detection of strokes such as ischaemic strokes.

Post contrast CT angiography can be used to obtain a series of cross-sectional post contrast CT images of the brain, e.g. by performing a sweeping scan up from the neck to the top of the head of the patient. The post contrast CT images are obtained over a short period during the arterial phase once the contrast has reached the brain. A radiologist or other skilled medical professional will manually inspect the series of images representing a single time point to observe the vessels of the brain which are opaque due to the presence of contrast, and identify any which appear occluded. This is achieved by careful inspection of the intracranial vessels, in particular by searching for vessels which appear to abruptly terminate at a location that is not seen in normality. This process is time consuming as the radiologist may have to sift through hundreds of images of the brain looking for subtle indications of narrowing or occlusion of the vessels. In addition, small occlusions indicative of small ischaemic strokes are hard to identify using post contrast CT angiography meaning that small strokes can be missed or misdiagnosed even by experienced radiologists.

Another problem with post contrast CT angiography is that the post contrast CT images obtained represent a single time point. As such, post contrast CT angiography is ineffective in assessing the patient's collaterals, i.e. a network of minor vessels in the brain which serve to re-route blood circulation around a blocked artery or vein. A patient with good collaterals will have a pathway for blood to get around a blocked vessel and supply the tissue which is being deprived of blood. Patients with good collaterals are better candidates for certain types of therapies, such as catheter directed thrombectomy.

Multiphase CT angiography is an extension of CT angiography that was developed to assess patient's collaterals. Multiphase CT angiography differs from CT angiography in that post contrast CT images of the brain are obtained at several (typically three different) time points, commonly referred to as phases, after the contrast is introduced into the body. The first phase is typically obtained during the arterial phase in the same way as normal CT angiography. The second phase is typically obtained around eight seconds after the first phase. The third phase is typically obtained around eight seconds after the second phase. Therefore, three CT images representing three different time points are typically obtained after a single injection of contrast. Using these different phases, the radiologist can observe the vessels of the brain over time and as such can assess the quality of the collaterals.

Multiphase CT angiography was not developed to improve the detection of ischaemic strokes in patients. Further, if multiphase CT angiography were used in stroke detection, it would still involve manually observing images to identify subtle indications of narrowing or occlusion of the vessels in much the same way as post contrast CT angiography. Small occluded intra-cranial vessels would be difficult to observe in the images, meaning that small ischaemic strokes could be missed or misdiagnosed.

It is an objective of the present invention to obviate and/or mitigate some or all of the problems identified above.

Accordingly, the present invention provides a method of processing cross-sectional post contrast images, the method comprising: obtaining a first cross-sectional post contrast image of at least part of a body, the first cross-sectional post contrast image representing a first time point; obtaining a second cross-sectional post contrast image of at least part of the body, the second cross-sectional post contrast image representing a second time point, the second time point being different from the first time point; and generating a first difference image using the first and second cross-sectional post contrast images, the first difference image highlighting any differences between the first and second cross-sectional post contrast images.

Here, “body” refers to a human, animal or plant body.

Here, “cross-sectional post contrast image” refers to an image of a cross-section of the body taken after contrast has been injected or otherwise introduced into the body. The contrast can be a radiocontrast agent, or other form of contrast dye as appropriately selected by the medical professional based on the desired imaging application. A common term for cross-sectional images is “slices”, which are typically obtained in the axial plane. Often sagittal and coronal plane slices are either reconstructed from the axial dataset, or other planes are individually acquired (such as in MRI). The cross-sectional post contrast images are not required to be obtained by a specific imaging technology. Acquiring images using, for example, magnetic resonance imaging (MRI) and computed tomography (CT) are within the scope of the present invention.

Here, “obtaining” may comprise obtaining the post contrast images during an imaging scan of the at least part of the body. Alternatively, “obtaining” may comprise obtaining the post contrast images from a data file or library, i.e. without requiring a new imaging scan to be performed on the at least part of the body. In this way, the present invention can be used with new or existing imaging data, and can be used to go back and check on previously acquired results.

Advantageously, the present invention generates a first difference image highlighting any differences between the first and second cross-sectional post contrast images. The first difference image aids in the detection of events which occur between the first and second time points. These events can result in subtle or small changes in opacification between the first and second time points which are difficult to observe from the first and second cross-sectional post contrast images themselves. The first difference image highlights any features not shared by the first and second cross-sectional post contrast images, making them easier to observe/detect. The problem of having a skilled operator carefully inspect/compare all of the post contrast images is avoided or mitigated. The present invention therefore provides a surprising new use of cross-sectional post contrast images obtained at different time points.

Advantageously still, the method does not require any changes to the image acquiring operation and instead provides substantial benefits just by improving the post-processing of the obtained images. This means that changes to an image processing apparatus, which must be carefully calibrated, can be avoided.

Preferably, the first difference image comprises performing a subtraction operation on the first and second cross-sectional post contrast images. The subtraction operation may comprise subtracting the first cross-sectional post contrast image from the second cross-sectional post contrast image. The subtraction operation may comprise subtracting the second cross-sectional post contrast image from the first cross-sectional post contrast image. In this way, the first difference image highlights any differences between the first and second cross-sectional post contrast images.

Here, subtracting the first cross-sectional post contrast image from the second cross-sectional post contrast image may mean that the pixel intensity values of pixels in the first cross-sectional post contrast image are subtracted from the pixel intensity values of correspondingly located pixels in the second cross-sectional post contrast image. For example, if the pixel at location (x1, y1) in the first cross-sectional post contrast image has a pixel intensity value of 10 and the pixel at location (x1, y1) in the second cross-sectional post contrast image has a pixel intensity value of 5, then the first difference image will have a pixel intensity value of −5 at location (x1, y1).

Here, subtracting the second cross-sectional post contrast image from the first cross-sectional post contrast image may mean that the pixel intensity values of pixels in the second cross-sectional post contrast image are subtracted from the pixel intensity values of correspondingly located pixels in the first cross-sectional post contrast image. For example, if the pixel at location (x1, y1) in the second cross-sectional post contrast image has a pixel intensity value of 5 and the pixel at location (x1, y1) in the first cross-sectional post contrast image has a pixel intensity value of 10, then the first difference image will have a pixel intensity value of 5 at location (x1, y1).

The first time point may be a predetermined time after the contrast is introduced into the body. The first time point may be selected based on the expected time required for contrast introduced into the body to reach the part of the body being imaged. The expected time can be known based on previously published data or may be calculated by introducing a small ‘test’ dose of contrast into the body and timing how long it takes to reach the part of the body being imaged. The arterial phase for the neck and head is typically 30 seconds from when contrast has been injected intravenously.

Preferably, the second time point is after the first time point. The second time point may be selected based on the expected time required for a difference in contrast opacification to develop in the body after the first time point. In this way, the second cross-sectional post contrast image will be expected to contain a difference from the first cross-sectional post contrast image. The second time point may be four seconds or more after the first time point. The second time point may be eight seconds or more after the first time point. In some applications such as when the part of the body being imaged is a kidney or kidneys, the second time point may be between ten and thirty minutes after the first time point.

Preferably, the body may be a patient potentially suffering from one or more vessel occlusions in a part of their body. The first and second cross-sectional post contrast images may be of the part of the body, and the first difference image may highlight the presence or absence of any occluded vessels in the part of the body.

Advantageously, the first difference image enables a medical professional to quickly confirm the presence of any occluded vessels which may be indicative of the patient suffering from ischaemia, i.e. a vascular disease involving an interruption in the arterial blood supply to a tissue, organ or extremity. The first difference image enables a medical professional to identify the presence of occlusions in small vessels which can be very difficult to identify from the cross-sectional post contrast images themselves. In addition, the first difference image identifies larger occlusions to a greater effect, increasing the speed and confidence with which a medical professional can make a diagnosis.

Advantageously still, if the difference image highlights zero or only a few insignificant differences between the first and second cross-sectional post contrast images, it helps provide a clear indication to the medical professional that the patient does not have occluded vessels, and other potential causes for the patient's condition should be considered.

The present invention is not limited to obtaining images from a particular part of the body. The part of the body may be the abdomen such that the first difference image may highlight the presence of vessel occlusions in the abdomen. The first difference image may therefore help a medical professional confirm the presence of a vascular disease such as mesenteric ischaemia in the abdomen. The part of the body may be an extremity, such as a lower limb. The first difference image may highlight the presence of vessel occlusions in the lower limb, which may therefore help a medical professional confirm the presence of a vascular disease such as lower limb ischaemia. The part of the body may be a kidney or kidneys such that the first difference image may highlight the presence of vessel occlusions in the kidney or kidneys. The first difference image may therefore help a medical professional confirm the presence of a vascular disease such as renal ischaemia.

Most preferably, the body is a patient potentially suffering from an acute ischaemic stroke. The first and second cross-sectional post contrast images may be of a section of the brain of the patient, and the first difference image may highlight the presence or absence of any occluded vessels in the section of the brain.

Advantageously, the first difference image highlights the presence of any occluded vessels in the section of the brain. This enables a medical professional to quickly confirm that the patient is having an acute ischaemic stroke and identify the location of the stroke in the brain, even if only a small vessel is occluded. Accordingly, the generation of the first difference image avoids the need for the medical professional to carefully analyse hundreds of cross-sectional post contrast images in order to try and detect small blocked vessels. The small blocked vessels are often missed in current clinical practice due to their difficulty to detect and the time pressures that medical professionals are under. The first difference image helps the medical professional diagnose whether the patient is experience an acute stroke syndrome or not.

The first and second cross-sectional post contrast images may be of substantially the same region of the body. The first and second cross-sectional post contrast images may sufficiently overlap/be aligned such that a separate post-processing alignment operation is not required. Alternatively, the method may further comprise aligning the first cross-sectional post contrast image with the second cross-sectional post contrast images.

Aligning the first cross-sectional post contrast image with the second cross-sectional post contrast image may comprise a user manually identifying at least one, and preferably at least three, common points in the first cross-sectional post contrast image and the second cross-sectional post contrast image. The method may further comprise aligning the first cross-sectional post contrast image with the second cross-sectional post contrast image based on the at least one common point. The at least one common point may be an anatomical feature present in the first and second cross-sectional post contrast images. Alternatively, the method may comprise automatically aligning the first and second cross-sectional post contrast images. Automatically aligning the first and second cross-sectional post contrast images may comprise detecting similarities between the first and second cross-sectional post contrast images and aligning the first and second cross-sectional post contrast images based on the detected similarities.

Preferably, the first difference image may be suitable for display to a user, such as a medical professional. The method may comprise displaying at least part of or all of the first difference image to the user. For example, only relevant parts of the difference image, i.e., which contain the differences between the first and second cross-sectional post contrast images can be displayed to the user. The first difference image may be displayed separately from or simultaneously with the first and/or second cross-sectional post contrast images.

The method may further comprise performing one or more post-processing operations on the first difference image. The post-processing operations may be for increasing the clarity of the first difference image. The post processing operations may include smoothing the first cross-sectional post contrast image and/or second cross-sectional post contrast image and/or first difference image to reduce image noise.

In some embodiments, obtaining the first and second cross-sectional post contrast image may comprise obtaining a plurality of first and second cross-sectional post contrast images representing the first or second time point. The plurality of first and second cross-sectional post contrast images may be generally aligned along the X and Y axes, and spatially separated along the Z axis. In other words, there are a plurality of groups of first and second cross-sectional post contrast images with each group representing a point along the Z axis. A first difference image may be obtained for each of the plurality of groups so that there are a plurality of first difference images. The plurality of first difference images may be grouped into slabs of predetermined thickness in the Z direction. A single representative first difference image may be obtained for one or more of the slabs. The single representative first difference image may be obtained by determining the maximum intensity projection (MIP) for one or more of the slabs. The slab thickness may be 25 mm. Other slab thicknesses could be appropriately selected by those skilled in the art.

Preferably, the first and/or second cross-sectional post contrast images are post contrast CT images. Advantageously, the present invention improves the detection of small vessel occlusions using CT imaging technology. This advantage is significant because while CT is preferred for stroke detection due to its wide availability and quick acquiring of images, it lags behind MRI in terms of image quality and ability to aid in detecting small vessel occlusions/acute strokes. The present invention helps overcome this disadvantage of CT imaging.

Obtaining the first and/or second cross-sectional post contrast images may comprise loading first and/or second cross-sectional post contrast images pre-stored on a memory device.

Obtaining the first and/or second cross-sectional post contrast images may comprise imaging the body at the first time point and the second time point. The first difference image may be generated automatically after obtaining the first and/or second cross-sectional post contrast images. Advantageously, this enables the method of the present invention to be used in rapid on-site evaluation of patients. The first and/or second cross-sectional post contrast images may be sent to a Picture Archive and Communication System (PACS).

Preferably, the method further comprises obtaining a third cross-sectional post contrast image of the body. The third cross-sectional post contrast image may represent a third time point, the third time point being different from the first and second time points.

The third time point may be after the first and second time points. The third time point may be selected based on the expected time required for a difference in contrast opacification to develop in the body after the first time point or the second time point. The third time point may be four seconds or more after the second time point. The third time point may be eight seconds or more after the second time point. In some applications such as when the part of the body being imaged is a kidney or kidneys, the third time point may be between ten and thirty minutes after the second time point.

The third cross-sectional post contrast image may be expected to contain a difference from the first or the second cross-sectional post contrast image.

The method may comprise generating a second difference image using the first and third cross-sectional post contrast images. The second difference image may highlight any differences between the first and third cross-sectional post contrast images.

The method may comprise generating a second difference image using the second and third cross-sectional post contrast images. The second difference image may highlight any differences between the second and third cross-sectional post contrast images.

The method may comprise generating second difference images using the first and third cross-sectional post contrast images and the second and third cross-sectional post contrast images.

The skilled person will appreciate that any or all of the above embodiments discussed in relation to the first and/or second cross-sectional post contrast images can be equally applied to the third cross-sectional post contrast image.

Preferably, the method further comprises generating an addition image using the first and second difference images. Generating the addition image may comprise adding the first difference image to the second difference image.

Advantageously, adding the first and second difference images together can convey information to the medical professional which is not directly discernible from the cross-sectional post contrast images and/or the difference images. For example, the addition image can be used to create a parenchymal difference image showing areas of the parenchyma which are under perfused. In particular, the addition image may display delayed parenchymal enhancement, which may be useful in the diagnosis of mesenteric ischaemia. The addition image may be advantageous in demonstrating the degree with which the bowel of a patient is at risk of becoming necrotic. Further, the addition image may be beneficial in pulmonary embolism imaging as the addition image may demonstrate the volume of the lung that is being compromised by the occlusion. In addition, if the embolic occlusion in to an extremity is very distal the actual occlusion may be too difficult to discern from the cross-sectional post contrast images/difference images, but the additional image showing the zone of parenchymal (or tissue) hypoenhancement may be more obvious.

The method may further comprise performing post-processing operations on the addition image. The post-processing operations may comprise performing a smoothing operation on the addition image, which may comprise using a Gaussian filter. The post-processing operations may comprise improving the contrast difference in the images. This is advantageous as the differences in tissue enhancement between normal brain and delayed perfused brain can be quite subtle. Improving the contrast difference in the images may comprise using a colour look-up table to map the grayscale values of the addition image to different colours. Typically, different colour look-up tables will be provided and the operator will select the most appropriate colour look-up table based on the obtained images. The contrast enhanced addition image may be displayed to the skilled medical professional. Other post-processing operations could be appropriately practised by those skilled in the art.

In addition to the first, second and third cross-sectional post contrast images, additional cross-sectional post contrast images representing different time points may be obtained without departing from the scope of the present invention. The additional cross-sectional post contrast images may be used to generate additional difference images and/or additional addition images. The number of cross-sectional post contrast images and subsequent difference images/addition images obtained/generated may be selected as appropriate by those skilled in the art based on the body to be imaged, and the type of feature intended to be observed in the difference images.

Accordingly, the present invention further provides an image processing system for carrying out any or all of the methods outlined above.

The image processing system may comprise a computing device. The computing device may have one or more processors, for example for carrying out one or more of the methods or parts thereof discussed above, particularly the processing of the first and second cross-sectional post contrast images.

The image processing system may comprise an imaging device operatively connected to the computing device.

The imaging device and the computing device may be integrated together.

Accordingly, the present invention further provides a computer-readable medium having computer executable code for carrying out any or all of the methods outlined above.

By way of example, a specific embodiment of the invention will now be described, with reference to the accompanying drawings, in which:

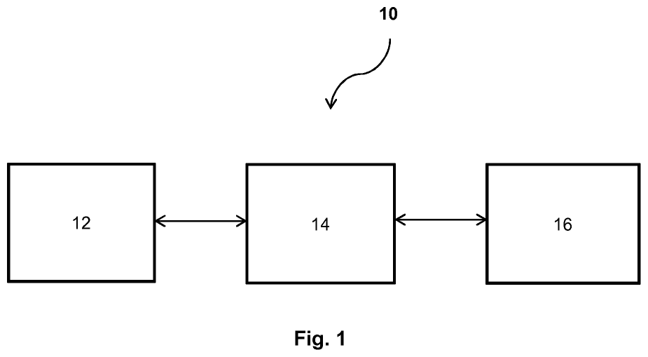

FIG. 1 is a schematic diagram of an image processing system according to an example embodiment;

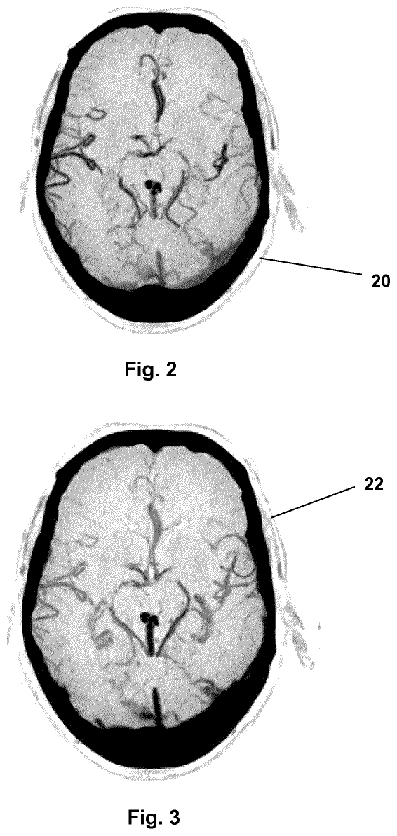

FIG. 2 is an example of a first cross-sectional post contrast image according to an example embodiment;

FIG. 3 is an example of a second cross-sectional post contrast image according to an example embodiment;

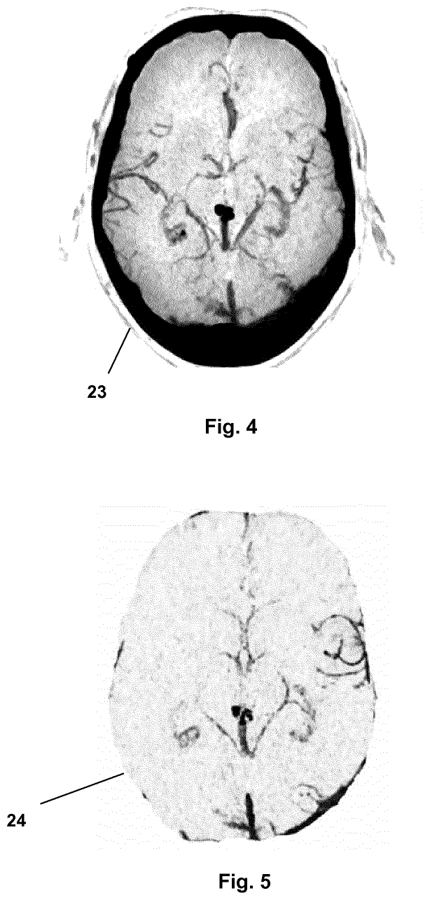

FIG. 4 is an example of a third cross-sectional post contrast image according to an example embodiment;

FIG. 5 is an example of a first difference image according to an example embodiment;

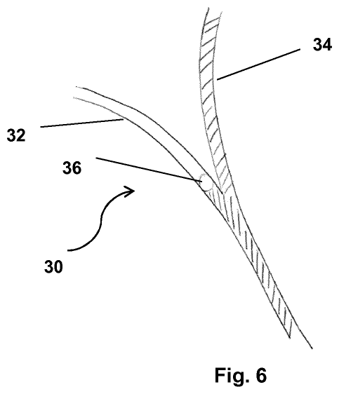

FIG. 6 is a simplified diagrammatic representation of an occluded vessel in the first cross-sectional post contrast image according to an example embodiment;

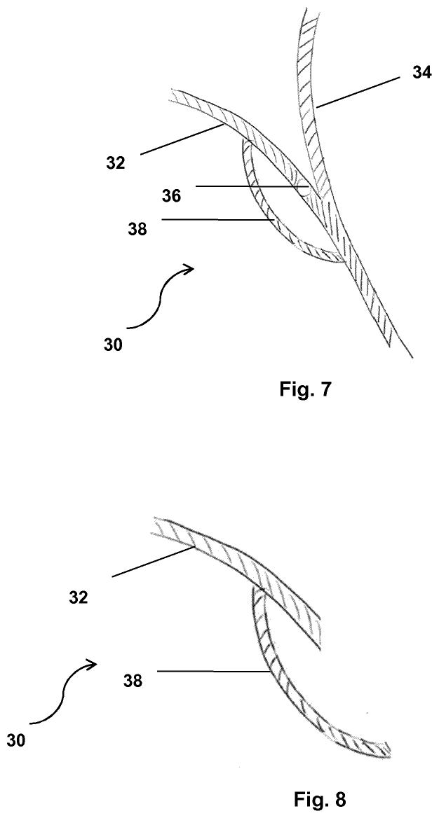

FIG. 7 is a simplified diagrammatic representation of the occluded vessel in the second cross-sectional post contrast image according to an example embodiment;

FIG. 8 is a simplified diagrammatic representation of the occluded vessel in the first difference image according to an example embodiment;

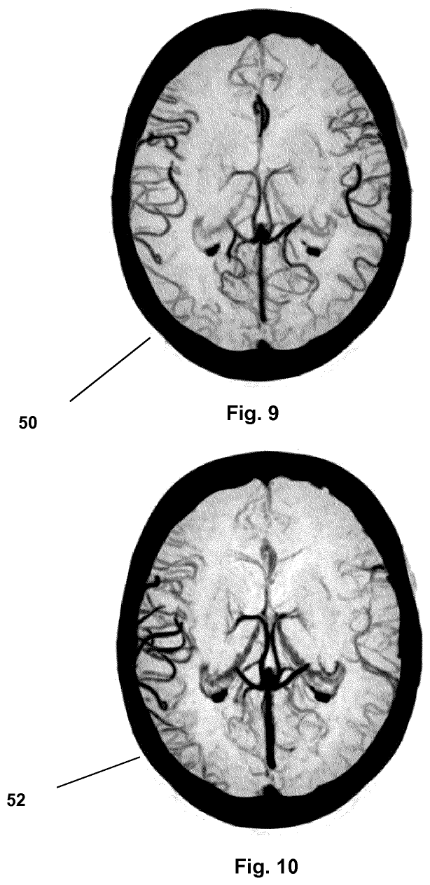

FIG. 9 is another example of a first cross-sectional post contrast image according to an example embodiment;

FIG. 10 is another example of a second cross-sectional post contrast image according to an example embodiment;

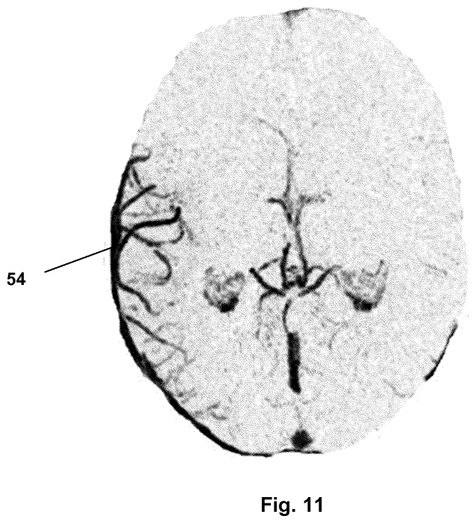

FIG. 11 is another example of a first difference image according to an example embodiment;

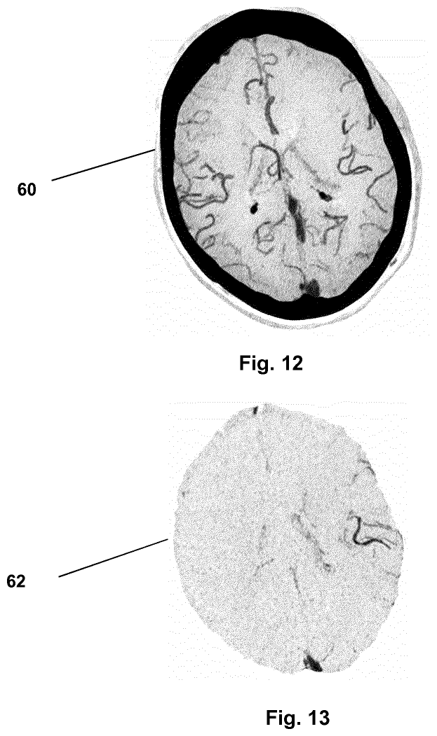

FIG. 12 is yet another example of a first cross-sectional post contrast image according to an example embodiment;

FIG. 13 is yet another example of a first difference image according to an example embodiment;

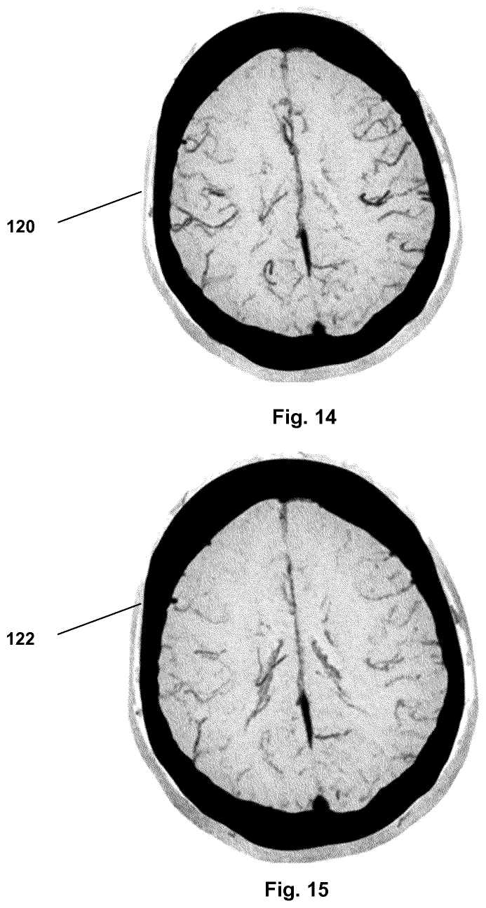

FIG. 14 is yet another example of a first cross-sectional post contrast image according to an example embodiment;

FIG. 15 is yet another example of a second cross-sectional post contrast image according to an example embodiment;

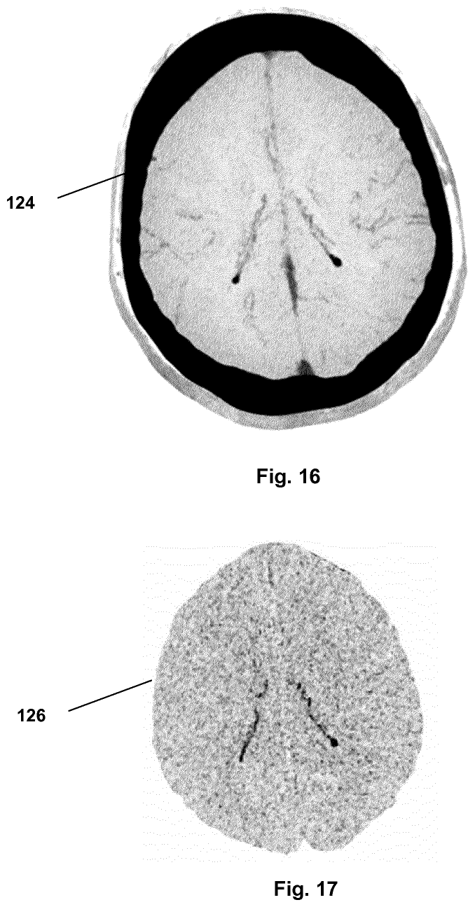

FIG. 16 is yet another example of a third cross-sectional post contrast image according to an example embodiment;

FIG. 17 is yet another example of a first difference image according to an example embodiment;

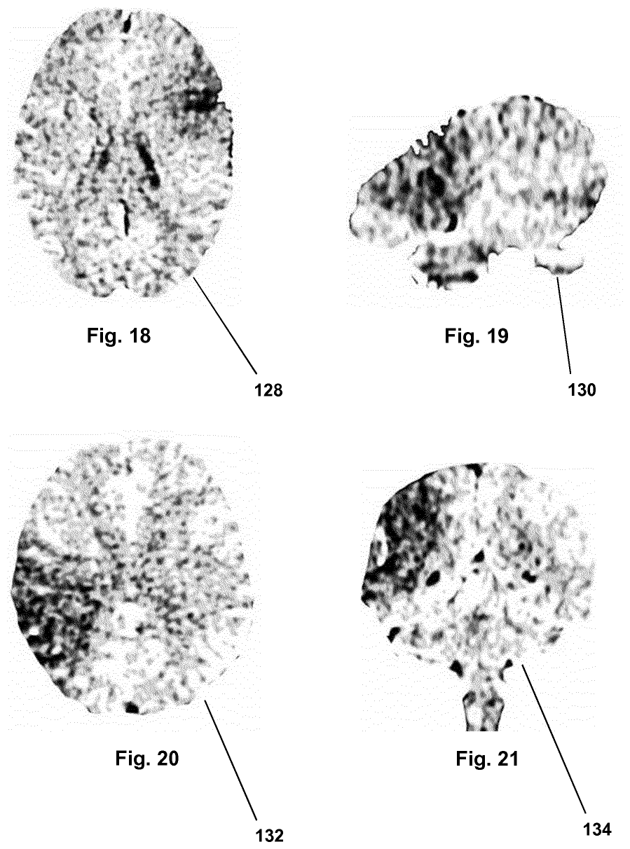

FIG. 18 is an example of an addition image according to an example embodiment;

FIG. 19 is another example of an addition image according to an example embodiment;

FIG. 20 is yet another example of an addition image according to an example embodiment;

FIG. 21 is yet another example of an addition image according to an example embodiment;

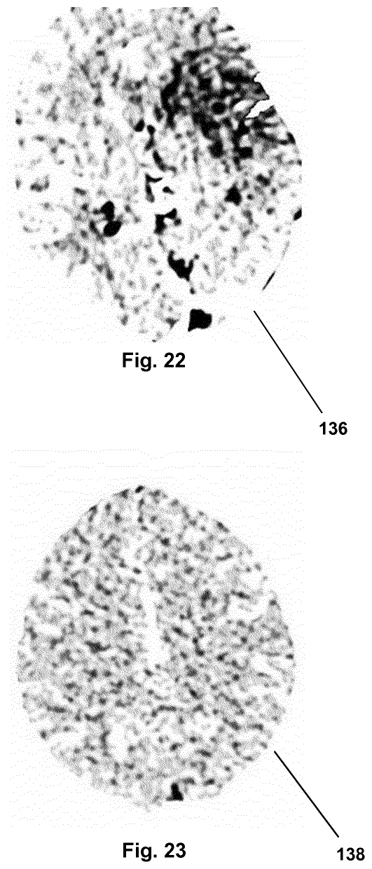

FIG. 22 is yet another example of an addition image according to an example embodiment;

FIG. 23 is yet another example of an addition image according to an example embodiment;

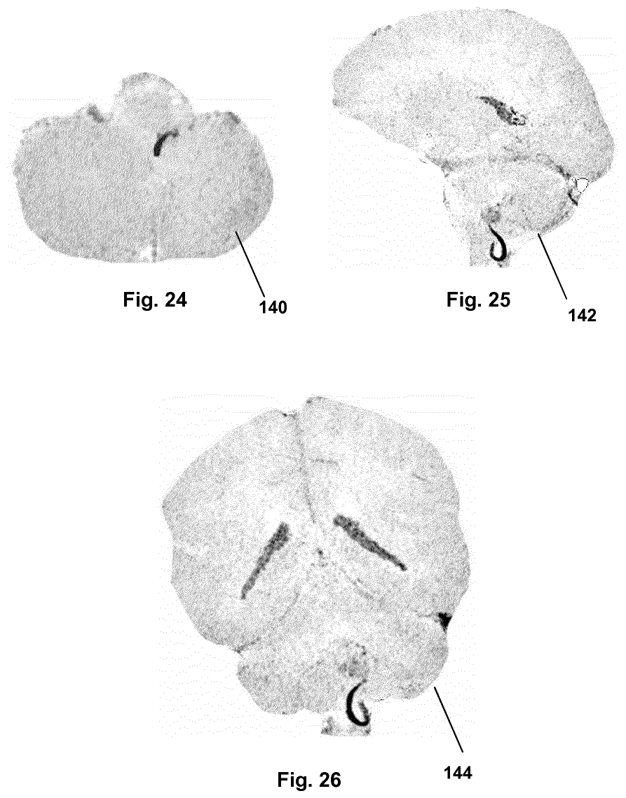

FIG. 24 is an example of a first difference image in the axial plane;

FIG. 25 is an example of a first difference image in the sagittal plane;

FIG. 26 is an example of a first difference image in the coronal plane;

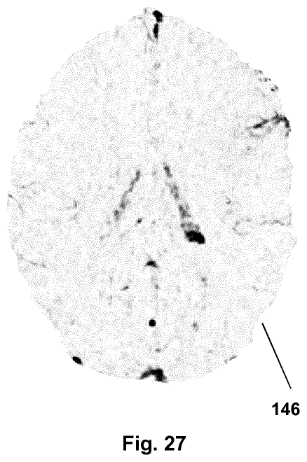

FIG. 27 is an example of a second difference image according to an example embodiment;

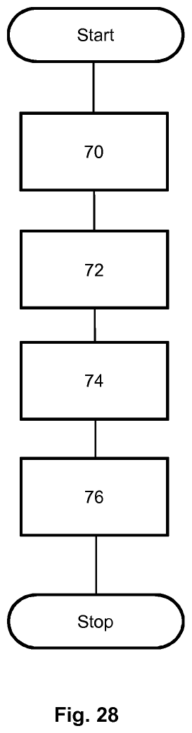

FIG. 28 is a diagrammatic representation of a method according to an example embodiment;

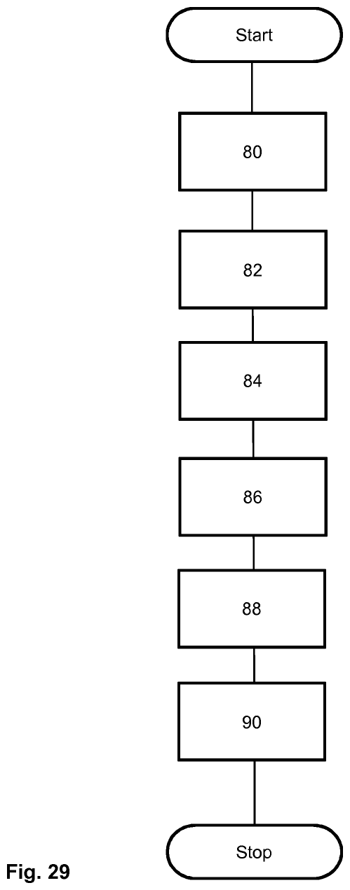

FIG. 29 is a diagrammatic representation of another method according an example embodiment;

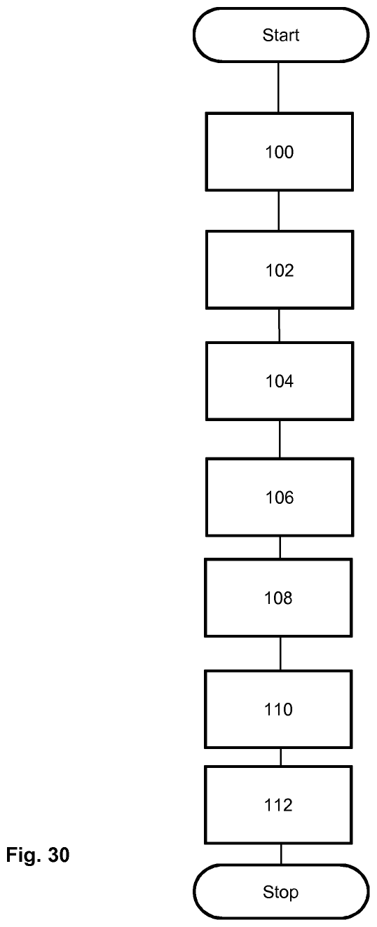

FIG. 30 is a diagrammatic representation of yet another method according to an example embodiment; and

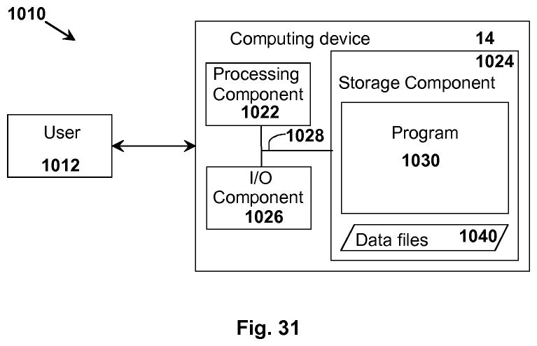

FIG. 31 is an illustrative environment according to an according to an example embodiment.

Referring to FIG. 1 there is shown an image processing system indicated generally by the reference numeral 10. The image processing system 10 comprises a CT scanner 12, a computing device 14 operatively connected to the CT scanner 12, and a display 16 operatively connected to the computing device. The CT scanner 12 is adapted to acquire a first cross-sectional post contrast image 20 of at least part of the body. The first cross-sectional post contrast image 20 represents a first time point. The CT scanner 12 is adapted to acquire a second cross-sectional post contrast image 22 of the at least part of the body. The second cross-sectional post contrast image 22 represents a second time point, different from the first time point. The computing device 14 is adapted to obtain the first and second cross-sectional post contrast images 20, 22 from the CT scanner 12. The computing device 14 is adapted to generate a first difference image 24 using the first and second cross-sectional post contrast images 20, 22. The display device 16 is adapted to display the first and/or second cross-sectional post contrast images 20, 22 and/or the first difference image 24.

In some embodiments, the CT scanner 12 is adapted to acquire a third cross-sectional post contrast image 23 of the part of the body. The third cross-sectional post contrast image 23 represents a third time point. The computing device 14 is adapted to obtain the third cross-sectional post contrast image 23 from the CT scanner 12. The computing device 14 is further adapted to generate a second difference image 146 using the first and third cross-sectional post contrast images 20, 23 or the second and third cross-sectional post contrast images 22, 23.

Referring to FIG. 2 there is shown a first cross-sectional post contrast image 20. As with all other images present in the drawings, the colours of the first cross-sectional post contrast image 20 have been inverted for the sake of better reproducibility. The first cross-sectional post contrast image 20 is an axial slice through a patient's brain and represents a first time point in the arterial phase after a contrast agent has been injected into the vascular system of the patient.

Referring to FIG. 3 there is shown a second cross-sectional post contrast image 22. The second cross-sectional post contrast image 22 is an axial slice through the same region of the patient's brain as the first cross-sectional post contrast image 20. The second cross-sectional post contrast image 22 represents a second time point which is around eight seconds after the first cross-sectional post contrast image 20 was acquired

Referring to FIG. 4 there is shown a third cross-sectional post contrast image 23. The third cross-sectional post contrast image 23 is an axial slice through the same region of the patient's brain as the first and second cross-sectional post contrast images 20, 22. The third cross-sectional post contrast image 23 represents a third time point which is around eight seconds after the second cross-sectional post contrast image 22 was acquired.

In the first, second and third cross-sectional post contrast images 20, 22, 23 several vessels in the brain are opaque due to the presence of contrast flowing through them. The medical professional observing the images 20, 22, 23 will be looking for any indications of occlusions in the blood vessels in the images 20, 22, 23. This could be indicated by the presence of asymmetry between the blood vessels of the right and left hemispheres of the brain in the images 20, 22, 23. The patient being imaged in the images 20, 22, 23 does in fact have an occlusion in the left hemisphere of the brain (right side of image). This is known as a left sided infarct. The occlusion is a small vessel occlusion which is very difficult to spot in the images 20, 22, 23 because there is no strong indication of asymmetry between the right and left hemispheres in the images 20, 22, 23. In view of this, a skilled medical professional could, and have been known to, miss this vessel occlusion and therefore not diagnose the presence of a small acute ischaemic stroke.

Referring to FIG. 5 there is shown a first difference image 24 obtained by subtracting the first cross-sectional post contrast image 20 from the second cross-sectional post contrast image 22. In particular, the pixel intensity value of each pixel in the first cross-sectional post contrast image 20 is subtracted from the pixel intensity value of a correspondingly located pixel in the second cross-sectional post contrast image 22. The first difference image 24 highlights any differences between the first and second cross-sectional post contrast images 20, 22. In particular, the difference is clearly shown as the presence of collateral vessels in the left hemisphere of the brain (right side of image). The medical professional observing the first difference image 24 will see a clear asymmetry between the right and left hemispheres of the brain due to the presence of the collateral vessels in the left hemisphere. The first difference image 24 provides a clear indication to the medical professional of the presence of a small intra-cranial vessel occlusion in the left hemisphere of the brain. This will enable the medical professional to make a quick and confident diagnosis of an acute ischaemic stroke in the patient, and the medical professional will also be able to pinpoint the location of the vessel occlusion. In view of this, the medical professional will be able to administer appropriate medication for the patient.

By generating a first difference image 24 that highlights any features not shared by the first and second cross-sectional post contrast images 20, 22, the medical professional is able to quickly identify the presence and location of vessel occlusions. This provides a substantial benefit to the medical community as it enables them to diagnose and treat medical conditions such as strokes more quickly and accurately. In addition, small acute ischaemic strokes are less likely to be overlooked. Generating the difference image 24 provides a surprising and unexpected new application for cross-sectional post contrast images obtained at different time points. Typically, these existing cross-sectional post contrast images have been used for completely different reasons, e.g. to identify a collateral as it appears over time. No-one has realised that the same initial images can be used in a powerful and beneficial new way, as disclosed herein.

Referring to FIG. 5, the first difference image 24 additionally shows the presence of opaque vessels in the central region of the brain. The medical professional will understand that these opaque vessels are intracranial veins and will discount them when looking for vessel occlusions. This is because veins, by definition, enhance later than arteries they will typically be visible in the first difference image 24. The veins have a characteristic location and morphology and so are unlikely to be misinterpreted as a delayed enhancing artery by a medical professional. In addition, as the venous system becomes more evident in the first difference image 24 it has the ancillary benefit of enabling the medical professional to assess for clots in the venous system. For example, in patients who do not have a delayed enhancing artery due to stroke (i.e. they are not having an acute embolic stroke), the veins can be quickly and confidently assessed for abnormalities, in particular thrombosis.

Referring to FIG. 6, there is shown a simplified diagrammatic representation of an occluded vessel indicated generally by the reference numeral 30 at the first time point. The occluded vessel 30 is a bifurcated vessel having two branches 32, 34. One of the branches 32 is blocked by an obstruction 36. It will be appreciated that the obstruction 36 is simply for diagrammatic purposes and the vessel does not have to be blocked in this way. Due to the presence of the obstruction 36, the blood containing contrast represented by the hatched lines is only able to pass through the branch 34. In the first cross-sectional post contrast image 20 which represents the first time point, the branch 32 will not be visible as there is no contrast flowing through. If the vessel 30 is small, it will be difficult for a medical professional to identify its absence. Therefore, the medical professional may be unable to identify the presence of the occlusion from the first cross-sectional post contrast image 20.

Referring to FIG. 7 there is shown a simplified diagrammatic representation of the same occluded vessel 30 at the second time point. In the second time point, a collateral vessel 38 is illuminated by blood flowing through it. The flow through the collateral vessel 38 is slower which is why it was not illuminated by contrast at the first time point. This is because the collateral pathways are longer and narrower than normal arterial pathways and so take longer to opacify with contrast. The collateral vessel 38 bypasses the obstruction 36. The collateral vessel 38 therefore enables blood containing contrast to reach the branch 32, but more slowly. In the second cross-sectional post contrast image 22 which represents the second time point, the branch 32 and the collateral vessel 38 is observable because they contain contrast. However, when the vessel 30 is small the presence of the collateral vessel 38 may be hard to identify from the second cross-sectional post contrast image 22, particularly if it is located in close proximity to other vessels. Therefore, the medical professional may be unable to identify the presence of the occlusion from the second cross-sectional post contrast image 22.

Referring to FIG. 8 there is shown a simplified diagrammatic representation of the occluded vessel 30 as it appears in the first difference image 24. In particular, the branch 32 and the collateral vessel 38 are observable, but the branch 34 and other features which are shared between the first and second cross-sectional post contrast images 20, 22 are removed. This means that the first difference image 24 highlights the difference between the first and second cross-sectional post contrast images 20, 22. The medical professional can quickly identify that there is an occluded vessel 30. Even if the occluded vessel 30 is small, it will be identifiable in the first difference image 24 because features shared by the first and second cross-sectional post contrast images 20, 22 are removed. The medical professional will only see the differences between the two images 20, 22 and so is able to make a quick and accurate diagnosis.

Referring to FIG. 9, there is shown a first cross-sectional post contrast image 50 through the brain of another patient suffering from an acute ischaemic stroke. In particular, the patient has an occluded vessel in the right hemisphere of the brain (left side of image), but it is not clearly visible from the first cross-sectional post contrast image 50 representing the first time point. This is known as a right sided infarct.

Referring to FIG. 10, there is shown a second cross-sectional post contrast image 52 through the same region of the brain as in FIG. 9 taken at a second time point after the first cross-sectional post contrast image 50. The second cross-sectional post contrast image 52 shows a hint of asymmetry in the right hemisphere of the brain which a medical professional would take as an indication of the presence of an occluded vessel. The medical professional, however, would need to carefully observe the second cross-sectional post contrast image 52 to make this diagnosis which wastes valuable time and delays the patient's treatment. This is to the extent that such a difference might require a second opinion, or may even be overlooked/misinterpreted in a hectic situation or even when the images are later slowly reviewed after the immediate treatment plan has been decided.

Referring to FIG. 11, there is shown a first difference image 54 taken by subtracting the first cross-sectional post contrast image 50 from the second cross-sectional post contrast image 52. The asymmetry in the left hemisphere of the brain is clear from the first difference image 54. Therefore, the first difference image 54 enables the medical professional to make a quick and confident diagnosis of an acute ischaemic stroke in the right hemisphere of the brain (left side of image).

Referring to FIG. 12 there is shown a first cross-sectional post contrast image 60 through the brain of yet another patient suffering from acute ischaemic stroke. In particular, the patient has an occluded vessel in the left hemisphere of the brain (right side of image), but it is not clearly visible from the first cross-sectional post contrast image 60 representing the first time point.

Referring to FIG. 13 there is shown a first difference image 62 taken by subtracting the first cross-sectional post contrast image 60 of FIG. 12 from a second cross-sectional post contrast image (not shown) representing a second time point. In the first difference image 62 the occluded vessel in the left hemisphere of the brain (right side of image) is clearly visible. Therefore, the medical professional is able to make a quick and accurate diagnosis of acute ischaemic stroke in the left hemisphere of the brain.

Referring to FIG. 14 there is shown a first cross-sectional post contrast image 120 through the brain of yet another patient at a first time point.

Referring to FIG. 15 there is shown a second cross-sectional post contrast image 122 through the same region of the brain as in FIG. 14 but at a second time point after the first time point.

Referring to FIG. 16 there is shown a third cross-sectional post contrast image 124 through the same region of the brain as in FIGS. 14 and 15 but at a third time point after the first and second time points.

The patient of FIGS. 14 to 16 is not actually suffering from any stroke symptoms. In existing systems, a medical professional can only confirm this by carefully inspecting the first, second and third cross-sectional post contrast images 120, 122, 124. This is time consuming and runs the risk of a false stroke diagnosis. This is disadvantageous because the patient may be suffering from another, non-stroke related symptom, which needs to be quickly identified and treated. As explained above, the medication available for treating ischaemic strokes can be dangerous and thus should not be given to patients who are not suffering from ischaemic strokes.

Referring to FIG. 17 there is shown a first difference image 126 generated by subtracting the first difference image 120 from the second difference image 122. The first difference image 126 is essentially empty save for the presence of intracranial veins in the centre of an image which the medical professional would quickly discount as discussed above. In other words, the first difference image 126 does not highlight the presence of any asymmetry. By observing the first difference image 126 the medical professional would be able to quickly and confidently confirm that the patient is not suffering from stroke symptoms due to the absence of any indications of vessel occlusions. Therefore, the first difference image 126 is advantageously helpful in highlighting the absence or presence of vessel occlusions.

Referring again to FIG. 1, the computing device 14, in another embodiment, is further adapted to generate an addition image 128 using the first and second difference images.

The display device 16 is adapted to display the third cross-sectional post contrast image 23 and the second difference image and the addition image 128.

Referring to FIG. 18, there is shown an addition image 128 obtained by adding the first difference image 24 of FIG. 5 to a second difference image 146 of FIG. 27. The second difference image 146 was generated using the first cross-sectional post contrast image 20 of FIG. 2 and the third cross-sectional post contrast image 23 of FIG. 4. In particular, the first cross-sectional post contrast image 20 was subtracted from the third cross-sectional post contrast image 23. The addition image 128 is a parenchymal difference image 128 that shows the area of the parenchyma which is under perfused. The under perfused region is visible in the anterior left aspect of the brain (upper right corner of the image) as a darker shaded region. In actual addition images, the under perfused regions will be lighter but are present as a darker region in the drawings due to the fact that the colours have been inverted to provide better reproducibility. A medical professional will also likely apply a colour map to the addition images so that the under perfused regions are more clearly visible. The region in the anterior left aspect of the brain (upper right corner of the image) is under perfused due to the occlusion shown by the difference image 24 in FIG. 5. By observing the addition image 128 the medical professional can determine what areas of the brain are under perfused due to the presence of the occlusion and thus at risk of dying during the stroke event if adequate treatment is not provided. Therefore, generating the addition image 128 provides a substantive additional technical benefit for the medical professional. This is a much better/faster way of analysing medical images than existing techniques.

Referring to FIG. 19, there is shown an addition image 130 of the same brain as in FIG. 18 but taken through the sagittal plane. The addition image 130 can provide the medical professional with further information about what areas of the brain are under perfused.

Referring to FIG. 20, there is shown an addition image 132 obtained by adding the first difference image 54 of FIG. 11 to a second difference image (not shown) of the same region of the brain. The addition image 132 shows that the right hemisphere of the brain (left side of image) is under perfused and thus is at risk of dying. This corresponds to the location of the occlusion identified in the first difference image 54.

Referring to FIG. 21, there is shown an addition image 134 of the same brain as in FIG. 20 but taken through the coronal plane.

Referring to FIG. 22, there is shown an addition image 136 obtained by adding the first difference image 62 of FIG. 13 to a second difference image (not shown) of the same region of the brain. The addition image 136 shows that the anterior left aspect of the brain (upper right corner of the image) is under perfused and thus is at risk of dying. This corresponds to the location of the occlusion identified in the first difference image 62.

Referring to FIG. 23, there is shown an addition image 138 obtained by adding the first difference image 126 of FIG. 17 to a second difference image (not shown) of the same region of the brain. The addition image 138 does not show that any part of the brain is under perfused, which corresponds to the fact that no occlusion was identified in the first difference image 126.

Referring to FIG. 24 there is shown another example of a first difference image 140 of a patient taken in the arterial plane. The first difference image 140 highlights the presence of a left posterior inferior cerebellar artery (PICA) infarction (centre-right side of image).

Referring to FIG. 25 there is shown another first difference image 142 of the patient in FIG. 24 but taken in the sagittal plane.

Referring to FIG. 26 there is shown another first difference image 144 of the patient in FIGS. 24 and 25 but taken in the coronal plane. The medical professional can use the arterial, sagittal and coronal plane images to accurately locate the infarction.

Referring to FIG. 28 there is shown a process diagram for a method of processing cross-sectional post contrast images according to embodiments of the present invention.

Step 70 comprises obtaining the first cross-sectional post contrast image 20 of the at least part of the body. The first cross-sectional post contrast image 20 represents the first time point.

Step 72 comprises obtaining the second cross-sectional post contrast image 22 of the at least part of the body. The second cross-sectional post contrast image 22 represents the second time point.

Step 74 comprises generating the first difference image 24 using the first and second cross-sectional post contrast images 20, 22.

Step 76 comprises displaying the first difference image 24 or at least part thereof.

Referring to FIG. 29 there is shown a process diagram for another method of processing cross-sectional post contrast images according to embodiments of the present invention.

Step 80 comprises obtaining the first cross-sectional post contrast image 20 of the at least part of the body. The first cross-sectional post contrast image 20 represents the first time point.

Step 82 comprises obtaining the second cross-sectional post contrast image 22 of the at least part of the body. The second cross-sectional post contrast image 22 represents the second time point.

Step 84 comprises obtaining the third cross-sectional post contrast image 23 of the at least part of the body. The third cross-sectional post contrast image 23 represents the third time point.

Step 86 comprises generating the first difference image 24 using the first and second cross-sectional post contrast images 20, 22.

Step 88 comprises generating the second difference image 146 using the first cross-sectional post contrast image 20 and the third cross-sectional post contrast image 23.

Step 90 comprises displaying the first difference image 24 and/or the second difference image or at least part thereof.

Referring to FIG. 30 there is shown a process diagram for yet another method of processing cross-sectional post contrast images according to embodiments of the present invention.

Step 100 comprises obtaining the first cross-sectional post contrast image 20 of the at least part of the body. The first cross-sectional post contrast image 20 represents the first time point.

Step 102 comprises obtaining the second cross-sectional post contrast image 22 of the at least part of the body. The second cross-sectional post contrast image 22 represents the second time point.

Step 104 comprises obtaining the third cross-sectional post contrast image 23 of the at least part of the body. The third cross-sectional post contrast image 23 represents the third time point.

Step 106 comprises generating the first difference image 24 using the first and second cross-sectional post contrast images 20, 22.

Step 108 comprises generating the second difference image using the first cross-sectional post contrast image 20 and the third cross-sectional post contrast image 23.

Step 110 comprises generating the addition image using the first difference image 24 and the second difference image.

Step 112 comprises displaying the first difference image 24 and/or the second difference image and/or the addition image or at least part thereof.

Referring to FIG. 31 there is shown an illustrative environment 1010 according to an embodiment of the invention. The skilled person will realise and understand that embodiments of the present invention may be implemented using any suitable computing device 14, and the example system shown in FIG. 17 exemplary only and provided for the purposes of completeness only. To this extent, environment 1010 includes a computing device 14 that can perform a process described herein in order to perform an embodiment of the invention. In particular, computing device 14 is shown including a program 1030, which makes computing device 14 operable to implement an embodiment of the invention by performing a process described herein, e.g. using by one or more processors.

Computing device 14 is shown including a processing component 1022 (e.g., one or more processors), a storage component 1024 (e.g., a storage hierarchy), an input/output (I/O) component 1026 (e.g., one or more I/O interfaces and/or devices), and a communications pathway 1028. In general, processing component 1022 executes program code, such as program 1030, which is at least partially fixed in storage component 1024. While executing program code, processing component 1022 can process data, which can result in reading and/or writing transformed data from/to storage component 1024 and/or I/O component 1026 for further processing. Pathway 1028 provides a communications link between each of the components in computing device 14. I/O component 1026 can comprise one or more human I/O devices, which enable a human user 1012 to interact with computing device 14 and/or one or more communications devices to enable a system user 1012 to communicate with computing device 14 using any type of communications link. To this extent, program 1030 can manage a set of interfaces (e.g., graphical user interface(s), application program interface, and/or the like) that enable human and/or system users 1012 to interact with program 1030. Further, program 1030 can manage (e.g., store, retrieve, create, manipulate, organize, present, etc.) the data, such as a plurality of data files 1040, using any solution.

In any event, computing device 14 can comprise one or more general purpose computing articles of manufacture (e.g., computing devices) capable of executing program code, such as program 1030, installed thereon. As used herein, it is understood that “program code” means any collection of instructions, in any language, code or notation, that cause a computing device having an information processing capability to perform a particular action either directly or after any combination of the following: (a) conversion to another language, code or notation; (b) reproduction in a different material form; and/or (c) decompression. To this extent, program 1030 can be embodied as any combination of system software and/or application software.

Further, program 1030 can be implemented using a set of modules. In this case, a module can enable computing device 14 to perform a set of tasks used by program 1030, and can be separately developed and/or implemented apart from other portions of program 1030. As used herein, the term “component” means any configuration of hardware, with or without software, which implements the functionality described in conjunction therewith using any solution, while the term “module” means program code that enables a computing device 14 to implement the actions described in conjunction therewith using any solution. When fixed in a storage component 1024 of a computing device 14 that includes a processing component 1022, a module is a substantial portion of a component that implements the actions. Regardless, it is understood that two or more components, modules, and/or systems may share some/all of their respective hardware and/or software. Further, it is understood that some of the functionality discussed herein may not be implemented or additional functionality may be included as part of computing device 14.

When computing device 14 comprises multiple computing devices 14, each computing device 14 can have only a portion of program 1030 fixed thereon (e.g., one or more modules). However, it is understood that computing device 14 and program 1030 are only representative of various possible equivalent computer devices that may perform a process described herein. To this extent, in other embodiments, the functionality provided by computing device 14 and program 1030 can be at least partially implemented by one or more computing devices 14 that include any combination of general and/or specific purpose hardware with or without program code. In each embodiment, the hardware and program code, if included, can be created using standard engineering and programming techniques, respectively.

Regardless, when computing device 14 includes multiple computing devices 14, the computing devices 14 can communicate over any type of communications link. Further, while performing a process described herein, computing device 14 can communicate with one or more other computer systems using any type of communications link. In either case, the communications link can comprise any combination of various types of optical fibre, wired, and/or wireless links; comprise any combination of one or more types of networks; and/or utilize any combination of various types of transmission techniques and protocols.

In any event, computing device 14 can obtain data from files 1040 using any solution. For example, computing device 14 can generate and/or be used to generate data files 1040, retrieve data from files 1040, which may be stored in one or more data stores, receive data from files 1040 from another system, and/or the like.

Although a few preferred embodiments have been shown and described, it will be appreciated by those skilled in the art that various changes and modifications might be made without departing from the scope of the invention, as defined in the appended claims.

1. A method of processing cross-sectional post contrast images, the method comprising:
obtaining a first cross-sectional post contrast image of at least part of a body, the first cross-sectional post contrast image representing a first time point;
obtaining a second cross-sectional post contrast image of the at least part of the body, the second cross-sectional post contrast image representing a second time point, the second time point being different from the first time point; and
generating a first difference image using the first and second cross-sectional post contrast images, the first difference image highlighting any differences between the first and second cross-sectional post contrast images.

2. A method as claimed in claim 1, wherein generating the first difference image comprises performing a subtraction operation on the first and second cross-sectional post contrast images, the subtraction operation comprising subtracting the first cross-sectional post contrast image from the second cross-sectional post contrast image.

3. A method as claimed in claim 1, wherein the second time point is after the first time point.

4. A method as claimed in claim 1, wherein the second time point is selected based on the expected time required for a difference in contrast opacification to develop in the body after the first time point.

5. A method as claimed in claim 1, wherein the first and second cross-sectional post contrast images are computed tomography (CT) post contrast images.

6. A method as claimed in claim 1, wherein the body is a patient potentially suffering from one or more vessel occlusions in a part of their body, the first and second cross-sectional post contrast images being of the part of the body, the first difference image highlighting the presence or absence of any occluded vessels in the part of the body.

7. A method as claimed in claim 6, wherein the patient is potentially suffering from an acute ischaemic stroke, the first and second cross-sectional post contrast images being of a section of the brain of the patient, the first difference image highlighting the presence or absence of any occluded vessels in the section of the brain.

8. A method as claimed in claim 1, further comprising aligning the first cross-sectional post contrast image with the second cross-sectional post contrast image.

9. A method as claimed in claim 1, further comprising obtaining a third cross-sectional post contrast image of the at least part of the body, the third cross-sectional post contrast image representing a third time point, the third time point being different from the first and second time points.

10. A method as claimed in claim 9, wherein the third time point is after the first and second time points.

11. A method as claimed in claim 9, further comprising generating a second difference using the first and third cross-sectional post contrast images, the second difference image highlighting any differences between the first and third cross-sectional post contrast images.

12. A method as claimed in claim 11, further comprising generating an addition image using the first and second difference images, generating the addition image comprises adding the first difference image to the second difference image.

13. An image-processing system comprising a computing device having at least one processor, the image processing system being configured to:
obtain a first cross-sectional post contrast image of at least part of a body, the first cross-sectional post contrast image representing a first time point;
obtain a second cross-sectional post contrast image of the at least part of the body, the second cross-sectional post contrast image representing a second time point, the second time point being different from the first time point; and
generate a first difference image using the first and second cross-sectional post contrast images, the first difference image highlighting any differences between the first and second cross-sectional post contrast images.

14. An image processing system as claimed in claim 13, further comprising an imaging device operatively connected to the computing device.

15. A computer-readable medium having computer executable code for
obtaining a first cross-sectional post contrast image of at least part of a body, the first cross-sectional post contrast image representing a first time point;
obtaining a second cross-sectional post contrast image of the at least part of the body, the second cross-sectional post contrast image representing a second time point, the second time point being different from the first time point; and
generating a first difference image using the first and second cross-sectional post contrast images, the first difference image highlighting any differences between the first and second cross-sectional post contrast images.

16. (canceled)
